(19) United States
(12) Patent Application Publication
(10) Pub. No.: US 2025/0340684 A1
(43) Pub. Date: Nov. 6, 2025

(54) METHOD OF MAKING HALO (ALKYL VINYL) ETHER MONOMERS AND FLUORINATED POLYMERS MADE WITH THE HALO (ALKYL VINYL) ETHER MONOMER

(71) Applicant: DUPONT SPECIALTY PRODUCTS USA LLC, WILMINGTON, DE (US)

(72) Inventors: DONALD LYONS, WILMINGTON, DE (US); ANILKUMAR RAGHAVANPILLAI, WILMINGTON, DE (US); MING-HONG HUNG, WILMINGTON, DE (US); MICHAEL COBB, WILMINGTON, DE (US)

(21) Appl. No.: 18/860,836

(22) PCT Filed: May 8, 2023
(86) PCT No.: PCT/US2023/021329

§ 371 Date: Oct. 28, 2024

Related U.S. Application Data
(60) Provisional application No. 63/342,704, filed on May 17, 2022.

(51) Int. Cl. C08F 16/24; C07C 43/17

(52) U.S. Cl. CPC C08F 16/24; C07C 43/17

(57) ABSTRACT

A method for making a halo (alkyl vinyl) ether comprising heating a reaction mixture comprising a combination of i) a metal; ii) a solvent; and iii) a halo (alkyl ethyl) ether according to formula (1) RCF₂OC(H)(X)CF₂Y (1) where R is independently H, F, Cl, Br, CF₂H, CF₃, CF₂CF₂H, linear perfluoroalkyl having 1 to 12 carbon atoms, or cyclic perfluoroalkyl having 1 to 12 carbon atoms, and X and Y are independently Cl, Br, I, or F, where X and Y are not both F; to form a reaction product mixture comprising a halo (alkyl vinyl) ether, the solvent, unreacted metal, and a metal salt.

### CROSS-REFERENCE TO RELATED APPLICATIONS

None

### FIELD OF THE INVENTION

The present invention relates, generally, to halo (alkyl vinyl) ether monomers, methods of making halo (alkyl vinyl) ether monomers, and fluorinated polymers made from the halo (alkyl vinyl) ether monomers.

### BACKGROUND OF THE INVENTION

Fluoropolymers such as fluoroelastomers are known to have excellent mechanical properties, heat resistance, weather resistance, and chemical resistance. Such properties make fluoroelastomers useful in many applications such as O-rings, seals, hoses, skid materials, and coatings (e.g., metal gasket coatings) that may be exposed to harsh environments including elevated temperature and corrosive chemicals. Parts made with fluoroelastomers find application in many industries including automotive, chemical processing, semiconductor, aerospace, and petroleum industries.

Fluoropolymers are made by the polymerization of fluoromonomers. One such fluoromonomer that has been used to make fluoroelastomers and fluororesins: is perfluoromethylvinylether (PMVE). PMVE has been polymerized to make homopolymers and copolymerized with other fluoromonomers to make different fluorinated copolymers.

Although PMVE is known to make fluoroelastomers with excellent properties, the properties of the fluoromonomers produced by PMVE, and fluoropolymers in general, can still be improved. For example, fluoropolymers with better heat resistance, weather resistance, chemical resistance, or improved physical properties such as increased or reduced flexibility are desired as compared to fluoropolymers made with PMVE.

Thus, additional halo (alkyl vinyl) ethers, different from PMVE, are of potential interest to create new fluoroelastomers with properties improved over prior fluoropolymers, such as those made with known monomers like PMVE. One such class of halo (alkyl vinyl) ethers of interest are partially perhalogenated (alkyl vinyl) ethers. However, these partially halogenated alkyl vinyl ethers have proven difficult to manufacture in quantities and yields that are commercially viable.

Therefore, a need exists for new methods of making partially halogenated (alkyl vinyl) ether monomers that may be used in the production of fluoroelastomers with potentially improved properties over known fluoropolymers.

### BRIEF SUMMARY OF THE INVENTION

The present invention is directed to a method for making a halo (alkyl vinyl) ether, comprising the steps of: heating a reaction mixture comprising a combination of i) a metal; ii) a solvent; and iii) a halo (alkyl ethyl) ether according to formula (1) RCF₂OC(H)(X)CF₂Y, where R is independently H, F, Cl, Br, CF₂H, CF₃, CF₂CF₂H, linear perfluoroalkyl having 1 to 12 carbon atoms, or cyclic perfluoroalkyl having 1 to 12 carbon atoms, and X and Y are independently Cl, Br, I, or F, where X and Y are not both F; to form a reaction product mixture comprising a halo (alkyl vinyl) ether, the solvent, unreacted metal, and metal salts.

The present invention is further directed to a fluoropolymer made by polymerizing the halo (alkyl vinyl) ether.

The present invention is still further directed to a fluoropolymer, comprising: a repeating unit (I)—[(C(H)(R¹)CF₂)]—, where R¹ is —OCRF₂, where R is independently H, F, Cl, Br, CF₂H, CF₃, CF₂CF₂H, linear perfluoroalkyl having 1 to 12 carbon atoms, or cyclic perfluoroalkyl having 1 to 12 carbon atoms.

The method of the invention allows for the halo (alkyl vinyl) ether to be produced at increased yields. The halo (vinyl ether) produced may be used to make new fluoropolymers with improved properties.

### DETAILED DESCRIPTION OF THE INVENTION

As used herein, the article “a” refers to one as well as more than one and does not necessarily limit its referent noun to the grammatical category of singular number.

As used herein, the term “article” refers to an unfinished or finished item, thing, object, or an element or feature of an unfinished or finished item, thing or object. As used herein, when an article is unfinished, the term “article” may refer to any item, thing, object, element, device, etc. that has a form, shape, configuration that may undergo further processing in order to become a finished article. When an article is unfinished, the term “preform” may refer to that form, shape, configuration, any part of which may undergo further processing to become finished. As used herein, when an article is finished, the term “article” refers to an item, thing, object, element, device, etc. that is in a form, shape, configuration that is suitable for a particular use/purpose without further processing of the entire entity or a portion of it.

An article may comprise one or more element(s) or subassembly (ies) that either are partially finished and awaiting further processing or assembly with other elements/subassemblies that together will comprise a finished article. In addition, as used herein, the term “article” may refer to a system or configuration of articles.

As used herein, the terms “comprises,” “comprising,” “includes,” “including,” “has,” “having” or any other variation of these, refer to a non-exclusive inclusion. For example, a process, method, article, or apparatus that comprises a list of elements is not limited to only the listed elements but may include other elements not expressly listed or inherent. Further, unless expressly stated to the contrary, “or” refers to an inclusive, not an exclusive, or. For example, a condition A or B is satisfied by any one of the following: A is true (or present) and B is false (or not present), A is false (or not present) and B is true (or present), and both A and B are true (or present).

As used herein, the terms “comprises,” “comprising,” “includes,” “including,” “has,” “having”, “consisting essentially of”, and “consisting of” or any other variation of these, may refer either to a non-exclusive inclusion or to an exclusive inclusion. When these terms refer to a more exclusive inclusion, these terms limit the scope of a claim to those recited materials or steps that materially affect the novel elements of the recited invention. When these terms refer to a wholly exclusive inclusion, these terms exclude any element, step or component not expressly recited in the claim.

As used herein, terms that describe molecules or polymers follow the terminology in the IUPAC Compendium of Chemical Terminology version 2.15 (International Union of Pure and Applied Chemistry) of Sep. 7, 2009.

As used herein, the term “alkyl” refers to linear, branched, or cyclic hydrocarbon structures and combinations of there. Alkyl does not include aromatic structures. Examples of linear alkyl groups include methyl, ethyl, propyl, butyl, pentyl, and hexyl groups. Branched alkyl groups include for example s- and t-butyl, and isopropyl groups. Examples of cyclic hydrocarbon groups include cyclopropyl, cyclobutyl, cyclopentyl, cyclohexyl, cycloheptyl, and cyclooctyl groups.

As used herein, the term “alkoxy” or “alkoxyl” refers to alkyl groups attached to an oxygen atom by a single bond. The other bond of the oxygen atom is connected to a carbon atom. Examples include methoxy, ethoxy, propoxy, isopropoxy, cyclopropyloxy, and cyclohexyloxy.

As used herein, the term “compound” refers to a composition that is able to be cured, i.e., a curable composition, as well as to a mixture of chemical entities that comprises at least a fluoroelastomer and a curing agent. The mixture of chemical entities has not been cured nor has undergone processing conditions that would cause the curing of the mixture of chemical entities to undergo curing.

As used herein, the prefix term “fluoro”, when placed as a prefix before a chemical entity name, refers to a chemical entity that has at least one fluorine atom as exemplified by the following designations: fluoroelastomers, perfluoroelastomers, fluorovinyl, and perfluorovinyl ethers. The prefix “fluoro”, when placed as a prefix before a chemical entity name, expressly includes “perfluoro” chemical entities. Thus, the prefix “fluoro”, when preceding a chemical entity name, indicates both “fluoro-” entities and “perfluoro-” entities.

As used herein, the term “cured” refers to that resultant entity that comprised a fluoroelastomer and which has been exposed to those conditions that caused the fluoroelastomer molecules to form sufficient crosslinks among themselves (that is, curing conditions) such that the resultant entity takes on a form or shape or configuration or structure that cannot be reprocessed, molded, or extruded into a different one. That is, once a resultant entity that comprised a fluoroelastomer has been exposed to curing conditions to thereby be cured, that entity cannot be re-cured in order to assume a substantially different form or structure.

As used herein, the term “curing” refers to that processing of a compound, also called herein curable composition, which results in an entity taking on a form or shape or configuration or structure that cannot be reprocessed, molded, or extruded into a different one. Such processing refers to the “curing process/processing”, which requires compounds to be exposed to certain conditions in order to initiate the curing process, such conditions called curing conditions.

The resultant entity of the curing process is a “cured” entity, that is, an article as defined hereinabove. To be clear, curing results in compounds taking on a form or shape or configuration or structure of an article. Cured articles of compounds described herein include, but are not limited to, O-rings, seals, and gaskets.

Compounds may be initially cured to achieve a non-reprocessable form, shape, etc., which has been termed “cured” herein. The cured compounds may be further subjected to additional curing conditions, which provide additional, subsequent curing. Such additional curing conditions may be variously termed herein either as “curing” or as “post-curing”. That is, the terms “curing”, “cured” refer to both an initial curing process that results in a first cured, resultant entity and also expressly refer to any subsequent curing process that results in a subsequently cured, resultant entity that may or not possess different material or physical properties than those of the first cured, resultant entity.

Any range set forth herein expressly includes its endpoints unless explicitly stated otherwise. Setting forth an amount, concentration, or other value or parameter as a range specifically discloses all possible ranges formed from any possible upper range limit and any possible lower range limit, regardless of whether such pairs of upper and lower range limits are expressly disclosed herein. Compounds, processes and articles described herein are not limited to specific values disclosed in defining a range in the description.

The disclosure herein of any variation in terms of materials, chemical entities, methods, steps, values, and/or ranges, etc.—whether identified as preferred or not—of the processes, compounds and articles described herein specifically intends to include any possible combination of materials, methods, steps, values, ranges, etc. For the purpose of providing photographic and sufficient support for the claims, any disclosed combination is a preferred variant of the processes, compounds, and articles described herein.

In this description, if there are nomenclature errors or typographical errors regarding the chemical name any chemical species described herein, the chemical structure takes precedence over the chemical name. And, if there are errors in the chemical structures of any chemical species described herein, the chemical structure of the chemical species that one of skill in the art understands the description to intend prevails.

A method for making a halo (alkyl vinyl) ether, comprising the following steps:

heating a reaction mixture comprising
i) a metal;
ii) a solvent; and
iii) a halo (alkyl ethyl) ether according to formula (1)

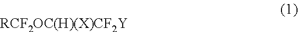

where R is independently H, F, Cl, Br, CF₂H, CF₃, CF₂CF₂H, linear perfluoroalkyl having 1 to 12 carbon atoms, or cyclic perfluoroalkyl having 1 to 12 carbon atoms, and X and Y are independently Cl, Br, I, or F, where X and Y are not both F;
to form a reaction product mixture comprising the halo (alkyl vinyl) ether, the solvent, unreacted metal, and metal salts.

A reaction mixture comprising a metal, a solvent, and a halo (alkyl ethyl) ether is heated to form a reaction product mixture comprising a halo (alkyl vinyl) ether, the solvent, unreacted metal, and metal salts.

The metal is any metal that will cause a dehalogenation reaction of the halo (alkyl ethyl) ether according to formula (1) in the solvent according to the invention, alternatively an alkali, alkaline earth, or transition metal group metal that will cause a dehalogenation reaction of the halo (alkyl ethyl) ether according to formula (1) in the solvent, alternatively the metal is zinc, magnesium, cadmium, or indium, alternatively zinc. Most metals are available commercially. One skilled in the art would know how to acquire and use the metal.

The solvent is a solvent sufficient for the dehalogenation reaction of the halo (alkyl ethyl) ether according to formula (1) with the metal, alternatively the solvent is an anhydrous polar aprotic solvent, alternatively the solvent is dimethylformamide (DMF), N-methylpyrolidone (NMP), Dimethylacetamide (DMAc), 1,3-Dimethyl-2-imidazolidinone (DMI), N,N′-Dimethylpropyleneurea (DMPU), Acetonitrile (MeCN), ethers or mixtures of two or more of dimethylformamide (DMF), N-methylpyrolidone (NMP), Dimethylacetamide (DMAc), 1,3-Dimethyl-2-imidazolidinone (DMI), N,N′-Dimethylpropyleneurea (DMPU), ethers, and Acetonitrile (MeCN). In one embodiment, the solvent is anhydrous. Examples of ethers include, but are not limited, to tetrahydrofuran (THF), dioxane, diglyme, triglyme, and tetraglyme. Many of the solvents that would function in the invention are available commercially.

The halo (alkyl ethyl) ether according to formula (1)

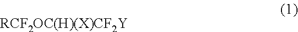

where R is H, F, Cl, Br, CF₂H, CF₃, CF₂CF₂H, linear perfluoroalkyl having 1 to 12 carbon atoms, or cyclic perfluoroalkyl having 1 to 12 carbon atoms, and X and Y are independently Cl, Br, I, or F, with the proviso that X and Y are not both F.

Examples of the halo (alkyl ethyl) ether according to formula (1) include, but are not limited to, 2-chloro-2-(difluoromethoxy)-1,1,1-trifluoro-ethane, 2-chloro-2-(chlorodifluoromethoxy)-1,1,1-trifluoro-ethane, 2-chloro-2-(bromodifluoromethoxy)-1,1,1-trifluoro-ethane, 2-chloro-2-(1-chloro-2,2,2-trifluoroethoxy)-1,1,2,2-tetrafluoro-ethane, 2-Chloro-2-(pentafluroethoxy)-1,1,1-trifluoro-ethane, 1,1,2,2,3,3-hexafluro-1-(2-chloro-1,1,1-trifluoroethoxy)-propane, 1-(1-Chloro-2,2,2-trifluoro)ethoxyperfluoropropane, 1-(1-Chloro-2,2,2-trifluoro)ethoxyperfluorobutane, 1-(1-Chloro-2,2,2-trifluoro)ethoxyperfluoroisobutane, 1-(1-Chloro-2,2,2-trifluoro)ethoxyperfluoro-sec-butane, 1-(1-Chloro-2,2,2-trifluoro)ethoxyperfluoropentane, 1-(1-Chloro-2,2,2-trifluoro)ethoxyperfluorohexane, 1-(1-Chloro-2,2,2-trifluoro)ethoxyperfluorocyclohexane, 1-(1-Chloro-2,2,2-trifluoro)ethoxyperfluorooctane, 1-(1-Chloro-2,2,2-trifluoro)ethoxyperfluorononane, 1-(1-Chloro-2,2,2-trifluoro)ethoxyperfluorodecane, 1-(1-Chloro-2,2,2-trifluoro). Many of the halogenatedethers according to formula (1) are available commercially or may be made by methods known in the art.

The halo (alkyl ethyl) ether is made by methods known in the art. Many halo (alkyl ethyl) ethers are available commercially.

The reaction product mixture comprises a halo (alkyl vinyl) ether, the solvent, unreacted metal, and metal salts.

The solvent and unreacted metal in the reaction product mixture are as described above for the reaction mixture.

The metal salts are halides formed from halogen from the halo (alkyl ethyl) ether and the metal. Examples of the metal salts include, but are not limited to, halides comprising fluorine, chlorine, and/or bromine and a metal comprising zinc, magnesium, cadmium, and/or indium metals such as zinc fluoride, zinc chloride, zinc bromide, magnesium fluoride, magnesium chloride, magnesium bromide, cadmium fluoride, cadmium chloride, cadmium bromide, indium fluoride, indium chloride, and indium bromide and mixtures thereof.

In one embodiment, the halo (alkyl vinyl) ether is according to formula (2)

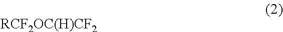

where R is H, F, Cl, Br, CF₂H, CF₃, CF₂CF₂H, linear perfluoroalkyl having 1 to 12 carbon atoms, or cyclic perfluoroalkyl having 1 to 12 carbon atoms; alternatively R is H, F, Cl, or Br, alternatively R is F.

Examples of the halo (alkyl vinyl) ether according to formula (2) include, but are not limited to, 2-(difluoromethoxy)-1,1-difluoroethene (HHPMVE), 1,1-difluoro-2-(trifluoromethoxy)ethene, 1,1-difluoro-2-(chlorodifluoromethoxy)ethene, 1,1-difluoro-2-(bromodifluoromethoxy)ethene, 1,1-difluoro-2-(1,1,2,2-tetrafluoroethoxy)ethene, 2-(pentafluoro)ethoxy-1,1-difloroethene, 1,1,2,2,3,3-hexafluro-1-(1,1-difluoroethenoxy)propane, 1-(1,1-difluoroethenoxy)perfluoropropane, 1-(1,1-difluoroethenoxy)perfluorobutane, 1-(1,1-difluoroethenoxy)perfluoroisobutane, 1-(1,1-difluoroethenoxy)perfluoro-sec-butane, 1-(1,1-difluoroethenoxy)perfluoropentane, 1-(1,1-difluoroethenoxy)perfluorohexane, 1-(1,1-difluoroethenoxy)perfluorocyclohexane, 1-(1,1-difluoroethenoxy)perfluorooctane, 1-(1,1-difluoroethenoxy)perfluorononane, 1-(1,1-difluoroethenoxy)perfluorodecane.

The reaction mixture is formed by combining the metal, solvent, and halo (alkyl ethyl) ether in a reaction vessel. One skilled in the art would know how to combine the components of the reaction mixture. Any reaction vessel known for use in dehalogenation reaction may be used. For example, the reaction vessel may be a steel reactor, a three-necked round bottom glass flask, a sealed tube reactor, or an autoclave with a cooling coil and pressure relief valve. The reaction vessel may or may not be equipped with means of stirring the reaction mixture. For example, the reaction vessel may be equipped with a magnetic stirrer. The reaction vessel is typically purged with a non-reactive gas prior to use.

The method of combining the components of the reaction mixture may vary. In one embodiment, there is no particular order of addition of the components of the reaction mixture to the reaction vessel. For example, the solvent may be added to the reaction vessel followed by the solvent then the halo (alkyl ethyl) ether, or the metal may be added first followed by the solvent and then the halo (alkyl ethyl) ether. In another embodiment, the metal and solvent are added to the reaction vessel prior to the halo (alkyl ethyl) ether. In one embodiment, the halo (alkyl ethyl) ether is added to the reactor last and the addition rate is controlled to control the reaction rate. One skilled in the art would know how to control the addition of the halo (alkyl ethyl) ether to control reaction rate.

The reaction mixture comprising the halo (alkyl ethyl) ether, the solvent, and the metal is heated. Methods known in the art for heating a reactor vessel may be used to heat the reaction mixture. For example, the reactor may be heated using a heating mantle or a furnace depending upon the type of reaction vessel used. One skilled in the art would know how to heat a reaction vessel.

The temperature at which the components of the reaction mixture are combined may vary. For example, the components of the reaction mixture may be combined at temperature below the temperature at which a reaction will readily occur, alternatively at a temperature from below ambient to ambient or slightly above ambient temperature, alternatively at a temperature below the boiling point of the components of the reaction mixture, alternatively from 0° C. to 30° C.

The temperature the reaction mixture is heated to may vary. Typically, the reaction mixture is heated to within a target temperature range, and then the reaction mixture temperature is held within that target temperature range until the reaction is sufficiently complete. One skilled in the art would know how to determine when the reaction is complete and how to heat a reaction mixture as discussed above. For example, the reaction mixture may be heated to raise the temperature of the reaction mixture at a rate of about 10° C./minute and the reaction may be monitored by chromatography.

The reaction mixture is heated to a temperature from 60° C. to 200° C., alternatively from 120° C. to 180° C., alternatively from 130° C. to 160° C.

The time at which the reaction mixture comprising the halo (alkyl ethyl) ether, metal, and solvent is held within the target temperature range may vary, alternatively the time is until the reaction is complete, or near complete, alternatively from 1 to 20 hours, alternatively 2-15 hours, alternatively from 4 to 10 hours. One skilled the art would know how to determine how long to hold the reaction mixture at the target temperature depending upon the progress of the reaction.

The solvent, the halo (alkyl ethyl) ether and the halo (alkyl vinyl) ether may have a boiling point above the target temperature range for the reaction, so the reaction may take place at elevated pressure. The pressure will vary depending upon the temperature of the reaction mixture, the pressure or partial pressure of the solvent, halo (alkyl ethyl) ether and halo (alkyl vinyl) ether in the product mixture. One skilled in the art would know how to determine the reaction pressure based upon the components of the reaction mixture and the reaction product mixture. Typically, no other means are taken to increase the pressure of the reaction mixture above the pressure generated from heating the components of the reaction mixture or the reaction product mixture. One skilled in the art would know the appropriate reactor to use at reaction pressures elevated above room pressure.

The weight ratio of the halo (alkyl ethyl) ether to the metal can vary, alternatively the weight ratio of halo (alkyl ethyl) ether to metal is from 1:0.01 to 1:0.75, alternatively 1:0.1 to 1:0.5, alternatively from 1:0.2 to 1:0.4. One skilled in the art would know how to determine the weight ratio of the halo (alkyl ethyl) ether to metal.

The amount of solvent in the reaction mixture comprising the halo (alkyl ethyl) ether, the solvent, and the metal can vary. In one embodiment, the solvent is from 10% (w/w) to 95% (w/w), alternatively 25% (w/w) to 80% (w/w), alternatively from 40% (w/w) to 70% (w/w), based on the weight of the solvent, halo (alkyl ethyl) ether, and the metal. One skilled in the art would understand how to optimize the amount of solvent in the reaction mixture.

The halo (alkyl vinyl) ether product may be recovered from the reaction product mixture. The reaction product mixture may be cooled, alternatively to about 25° C., alternatively to about 15° C., and any gases from the reaction vessel may be collected from the reactor using methods known in the art, for example a gas trap. The halo (alkyl vinyl) ether may be then recovered from reaction product mixture by methods known in the art. For example, the halo (alkyl vinyl) ether may be recovered using distillation or gas chromatography. One skilled in the art would recognize that there are different methods that may be used to recover the halo (alkyl vinyl) ether product.

In one embodiment of the invention, the halo (alkyl vinyl) ether is polymerized to form a fluoropolymer. The halo (alkyl vinyl) ether is as described above. The halo (alkyl vinyl) ether may be polymerized to form a homopolymer, or the halo (alkyl vinyl) ether may be polymerized with additional known monomers used to make fluoropolymers to form copolymers, terpolymers, and so forth.

The fluoropolymer may be cured, not curable, or curable, alternatively the fluoropolymer is curable, alternatively non-curable, alternatively cured. One skilled in the art would understand what a cured, non-curable, or curable fluoropolymer is and how to make a fluoropolymer that is non-curable or curable. In one embodiment, a curable fluoropolymer may be made by polymerizing the halo (alkyl vinyl) ether with a monomer comprising a cure site.

The fluoropolymer may be an amorphous or non-amorphous. Examples of amorphous fluoropolymers include, but are not limited to, a fluoroelastomer gum or a perfluoroelastomer gum made from the halo (alkyl vinyl) ether of the invention, a monomer with cure site, and additional monomers to give a gum with desirable properties.

Examples of the additional monomers that may be polymerized with the halo (alkyl vinyl) ether to form the fluoropolymer include, but are not limited to, perfluoroolefins, perfluoro(alkyl vinyl) ethers (PAVE) and perfluoroalkoxyalkyl vinyl ethers (PAAVE)), fluoro (alkene ether), halogenated fluoroolefins such as chlorotrifluoroethylene (CTFE)), partially fluorinated olefins such as vinyl fluoride (VF), vinylidene fluoride (VF2), trifluoroethylene, tetrafluoropropene (TFP), pentafluoropropene (HPFP), an olefin in which less than half or less than one-fourth of the hydrogen atoms are replaced with fluorine, an olefin according to the formula CX₂═CXR, where each X is independently hydrogen, fluoro, or chloro and R is hydrogen, fluoro, or a C₁-C₁₂, alternatively C₁ to C₃, alkyl, with the proviso that all X and R groups are not fluoro groups, hydrogen-containing monomers such as ethylene, propylene, and other non-fluorinated alpha-olefins such as a C₂ to C₉ alpha olefin, fluorinated ester ethers such as fluorinated estervinylether, methyl perfluoro(5-methyl-4,7-dioxanon-8-enoate) (EVE), or perfluoro(4-methyl-3,6-dioxaoct-7-ene)sulfonyl fluoride (PSEPVE), perfluoro(3-methoxypropyl vinyl ether) (MV-31), and nitrogen-containing cure site monomers. One skilled in the art would know where to find or how make the additional monomers, and many of these additional monomers are available commercially.

Examples of perfluoroolefins include, but are not limited to, tetrafluoroethylene (TFE), hexafluoropropylene (HFP), or any perfluoroolefin of the formula CF₂═CF—R_{f}, where R_{f} is fluorine or a perfluoroalkyl of 1 to 8, alternatively 1 to 3, carbon atoms,

Examples of PAAVE monomers include, but are not limited to, those according to the formula CF₂═CF—ORf, wherein Rf is a linear, branched, or cyclic perfluorinated alkyl group optionally containing ether linkages, and CF₂═CF(OCₙF₂ₙ)ₚORf, wherein Rf is a perfluorinated (C₁-C₈) alkyl group optionally containing ether linkages, each n is independently 1 to 4, and p is 1 to 6. When more than one CₙF₂ₙ group is present, “n” may be independently selected, alternatively n is from 1 to 12, alternatively 1 to 6. However, within a CₙF₂ₙ group, a person skilled in the art would understand that “n” is not independently selected. CₙF₂ₙ may be linear or branched. In some embodiments (OCₙF₂ₙ)ₚ is represented by —O—(CF₂)₁₋₄—[O(CF₂)₁₋₄]₀₋₁. Such perfluorinated ethers are described, for example, in U.S. Pat. Nos. 6,255,536 and 6,294,627 (each to Worm et al.) Examples of suitable PAAVE monomers include, but are not limited to, CF₂═CFOCF₂OCF₃, CF₂═CFOCF₂OCF₂CF₃, CF₂═CFOCF₂CF₂OCF₃, CF₂═CFOCF₂CF₂CF₂OCF₃ (MV-31), CF₂═CFOCF₂CF₂CF₂CF₂OCF₃, CF₂═CFOCF₂CF₂OCF₂CF₃, CF₂═CFOCF₂CF₂CF₂OCF₂CF₃, CF₂═CFOCF₂CF₂CF₂CF₂OCF₂CF₃, CF₂═CFOCF₂CF₂OCF₂OCF₃, CF₂═CFOCF₂CF₂OCF₂CF₂OCF₃, CF₂═CFOCF₂CF₂OCF₂CF₂CF₂OCF₃, CF₂═CFOCF₂CF₂OCF₂CF₂CF₂CF₂OCF₃, CF₂═CFOCF₂CF₂OCF₂CF₂CF₂CF₂CF₂OCF₃, CF₂═CFOCF₂CF₂(OCF₂)₃OCF₃, CF₂═CFOCF₂CF₂(OCF₂)₄OCF₃, CF₂═CFOCF₂CF₂OCF₂OCF₂OCF₃, CF₂═CFOCF₂CF₂OCF₂CF₂CF₃CF₂═CFOCF₂CF₂OCF₂CF₂OCF₂CF₂CF₃, CF₂═CFOCF₂CF(CF₃)—O—C₃F₇ (PPVE-2), CF₂═CF(OCF₂CF(CF₃))₂—O—C₃F₇ (PPVE-3), and CF₂═CF(OCF₂CF(CF₃))₃—O—C₃F₇ (PPVE-4). Methods of making PAAVE monomers are known in the art. Many of the PAAVE monomers are available commercially.

Examples of suitable PAVE monomers include, but are not limited to, perfluoro(methyl vinyl) ether CF₂═CFOCF₃, perfluoro(ethyl vinyl) ether CF₂═CFOCF₂CF₃, and perfluoro(n-propyl vinyl) ether CF₂═CFOCF₂CF₂CF₃. Mixtures of PAVE and PAAVE may also be employed. Methods of making PAVE monomers are known in the art. Many of the PAVE monomers are available commercially.

Examples of fluoro (alkene ether) monomers include, but are not limited to, those described in U.S. Pat. No. 5,891,965 (Worm et al.) and U.S. Pat. No. 6,255,535 (Schulz et al.). Such monomers include those represented by formula CF₂═CFCF₂(OCₙF_{2II})ₚORf, wherein n, p, and Rf are as defined above for the PAAVE monomers. Examples of suitable fluoro (alkene ether) monomers include perfluoroalkoxyalkyl allyl ethers such as CF₂═CFCF₂OCF₂CF₂OCF₃, CF₂═CFCF₂OCF₂CF₂CF₂OCF₃, CF₂═CFCF₂OCF₂OCF₃CF₂═CFCF₂OCF₂OCF₂CF₃, CF₂═CFCF₂OCF₂CF₂CF₂CF₂OCF₃, CF₂═CFCF₂OCF₂CF₂OCF₂CF₃, CF₂═CFCF₂OCF₂CF₂CF₂OCF₂CF₃, CF₂═CFCF₂OCF₂CF₂CF₂CF₂OCF₂CF₃, CF₂═CFCF₂OCF₂CF₂OCF₂OCF₃, CF₂═CFCF₂OCF₂CF₂OCF₂CF₂OCF₃, CF₂═CFCF₂OCF₂CF₂OCF₂CF₂CF₂OCF₃, CF₂═CFCF₂OCF₂CF₂OCF₂CF₂CF₂CF₂OCF₃, CF₂═CFCF₂OCF₂CF₂OCF₂CF₂CF₂CF₂CF₂OCF₃, CF₂═CFCF₂OCF₂CF₂(OCF₂)₃OCF₃, CF₂═CFCF₂OCF₂CF₂(OCF₂)₄OCF₃, CF₂═CFCF₂OCF₂CF₂OCF₂OCF₂OCF₃, CF₂═CFCF₂OCF₂CF₂OCF₂CF₂CF₃, CF₂═CFCF₂OCF₂CF₂OCF₂CF₂OCF₂CF₂CF₃, CF₂═CFCF₂OCF₂CF(CF₃)—O—C₃F₇, and CF₂═CFCF₂(OCF₂CF(CF₃))₂—O—C₃F₇. Many of these perfluoroalkoxyalkyl allyl ethers can be prepared, for example, according to the methods described in U.S. Pat. No. 4,349,650 (Krespan). Also, perfluoropropyl allyl ether (CF₂═CF—CF₂—OC₃F₇) and perfluoromethoxy ethyl allyl ether (CF2═CF—CF2-0C2F40CF3) can be prepared according to the methods described in U.S. Pat. No. 5,891,965 (Worm). Perfluoroalkoxyalkyl allyl ethers can also be prepared by combining first components comprising at least one of CF₂═CF—CF₂—OSO₂C₁ or CF₂═CF—CF₂—OSO₂CF₃, a polyfluorinated compound comprising at least one ketone or carboxylic acid halide or combination thereof, and fluoride ion. Polyfluorinated compounds comprising at least one ketone or carboxylic acid halide or combination thereof and fluoride ions can be any of those described, for example, in U.S. Pat. No. 4,349,650 (Krespan). Many fluoro (alkene ether) monomers such as perfluoroalkoxyalkyl allyl ether monomers are available commercially.

In one embodiment, the fluoropolymer comprises polymerized units derived from the halo (alkyl vinyl) ether according to formula (2), alternatively polymerized units derived from the halo (alkyl vinyl) ether according to formula (2) and one or more of the additional monomers TFE, PAVE, PAAVE, VF2, VF, and/or PMVE, alternatively one or more of TFE, VF2, and/or PMVE, alternatively TFE, alternatively VF2, alternatively PMVE, each of which polymers may also contain a monomeric unit having a nitrogen-containing cure site.

Liquid monomers including, but not limited to, the halo (alkyl vinyl) ether according to formula (2) used to make the fluoropolymer may be pre-emulsified with an emulsifier before polymerization with other monomers, for example, addition of a gaseous fluoroolefin.

The fluoropolymer comprises at least 0.5% (w/w), alternatively at least 1% (w/w), alternatively from 1% to 99.9% (w/w), alternatively from 1 to 50% (w/w), alternatively 1% to 20% (w/w) of polymerized units derived from fluoro (alkly vinyl) ether according to formula (2).

The fluoropolymer may comprise additional monomers, alternatively from 0 to 99.5% (w/w), alternatively 0% to 99% (w/w), alternatively 0.1% (w/w) to 99% (w/w), alternatively 80% (w/w) to 99% (w/w), alternatively from 50% to 99% (w/w), based on the weight of the fluoropolymer, of polymerized units derived from one or more of the additional monomers described above.

Examples of fluoropolymers include, but are not limited to, an HHPMVE homopolymer, an HPPMVE/TFE copolymer, an HHPMVE/VF2 copolymer, an HHPMVE/TFE/PMVE copolymer, and HHPMVE/VF2/PMVE copolymer, and HHPMVE/TFE/propylene copolymer, an HHPMVE/TFE/propylene/VF2 copolymer, an HHPMVE/VF2/HFP copolymer, an HHPMVE/TFE/VF2/HFP copolymer, an HHPMVE/TFE/CF₂═CFOC₃F₇ copolymer, an HHPMVE/TFE/CF₂═CFOCF₃/CF₂═CFOC₃F₇ copolymer, an HHPMVE/TFE/ethyl vinyl ether (EtVE) copolymer, an HHPMVE/TFE/butyl vinyl ether (BVE) copolymer, an HHPMVE/TFE/EtVE/BVE copolymer, an HHPMVE/VF2/CF₂═CFOC₃F₇ copolymer, an HHPMVE/ethylene/HFP copolymer, an HHPMVE/TFE/HFP copolymer, an HHPMVE/CTFE/VF2 copolymer, an HHPMVE/TFE/VF2 copolymer, an HHPMVE/TFE/VF2/PMVE/ethylene copolymer, and an HHPMVE/TFE/VF2/CF₂═CFO(CF₂)₃OCF₃ copolymer, each of which copolymers may also contain a monomeric unit having a nitrogen-containing cure site.

In some embodiments, the fluoropolymer contains units derived from TFE that have a molar ratio of units derived from TFE to comonomer units derived from the perfluoroalkyl vinyl or allyl ethers or perfluoroalkoxyalkyl vinyl or allyl ethers, and the halo (alkyl vinyl) ether according to formula (2) described above from, for example, 1:1 to 4:1, wherein the unsaturated ethers may be used as single compounds or as combinations of two or more of the unsaturated ethers. Typical compositions also comprise from 0.1-10% (w/w) of nitrogen-containing cure site monomers with the amount of ingredients being selected such that the total amount is 100% (w/w). Other typical compositions comprise from about 1-30% (w/w) of HHPMVE, 17-30 wt. % TFE, 25-38 wt. % VF2, 28-42 wt. % HFP and from 0.1-10% wt. nitrile-containing cure site monomers and from 0-10% wt. of other comonomers or modifiers with the amount of ingredients being selected such that the total amount is 100% wt.

Cure sites in the fluoropolymer enable curing the fluoropolymer to form cured fluoropolymers including a fluoroelastomer. A An example of a cure site component of a fluoropolymer comprises a nitrogen-containing group. Examples of monomers comprising nitrogen-containing groups useful in preparing fluoropolymers comprising a nitrogen-containing cure sites include free-radically polymerizable nitriles, imidates, amidines, amides, imides, and amine-oxides. Mixtures of any of these nitrogen-containing cure sites may be useful in the fluoropolymer compositions according to the present disclosure. Useful nitrogen-containing cure site monomers include nitrile-containing fluorinated olefins and nitrile-containing fluorinated vinyl ethers, for example, CF₂═CFO(CF₂)_{L}CN, CF₂═CFO(CF₂)ᵤOCF(CF₃)CN, CF₂═CFO[CF₂CF(CF₃)O]_{q}(CF₂O)_{y}CF(CF₃)CN, CF₂═CFO[CF₂FCF₃O]ₙCF₂—CFCF₃CN or CF₂═CF[OCF₂CF(CF₃)]_{q}O(CF₂)ₜCN, wherein L is in a range from 2 to 12; u is in a range from 2 to 6; q is in a range from 0 to 4; y is in a range from 0 to 6; n is in a range from 0 to 4; r is in a range from 1 to 2; and t is in a range from 1 to 4. Examples of such nitrogen-containing cure site monomers include CF₂═CFO(CF₂)₃OCF(CF₃)CN, perfluoro(8-cyano-5-methyl-3,6-dioxa-I-octene) (8-CNVE), and CF₂═CFO(CF₂) 5CN.

Nitrogen-containing cure sites can also be incorporated into the curable fluoropolymer by employing selected chain transfer agents (e.g., I(CF₂)_{d}CN in which d is 1 to 10 or 1 to 6) or by carrying out the free-radical polymerization in the presence of a perfluorosulfinate such as NC(CF₂)_{d}SO₂G, in which G represents a hydrogen atom or a cation with valence of 1 or 2.

The nitrogen-containing monomer, chain transfer agent, and/or initiator typically makes up about 0.1 to 5 mole percent (in some embodiments, 0.3 to 2 mole percent) of the polymerization components.

The fluoropolymers provided herein may further, or alternatively, comprise at least one halogen atoms cure site capable of participating in a peroxide cure reaction. The halogen capable of participating in a peroxide cure reaction can be bromine or iodine, alternatively iodine. The halogen atom capable of participating in the peroxide cure reaction may be located at a terminal position of the backbone chain, alternatively is located at a terminal position of the backbone chain. However, further reactive cure sites may also be present when the location of the halogen atom capable of participating in the peroxide cure reaction is located at a terminal position. The amount of iodine, bromine or the combination thereof contained in the fluoropolymer is between 0.001 and 5%, preferably between 0.01 and 2.5%, or 0.1 to 1% or 0.2 to 0.6% by weight with respect to the total weight of the fluoropolymer.

Examples of the cure site monomers capable of participating in the peroxide cure reaction when incorporated in the fluoropolymer include, but are not limited to, CF₂═CHBr, CH₂═CHCH₂Br, CF₂═CFCF₂Br, CH₂—═CHCF₂CF₂Br, CF₂═CHI, CH₂═CHCH₂I, CF₂═CFCF₂I, CH₂═CHCF₂CF₂I, CF₂═CFOC₄F₈I (MV4I), CF₂═CFOC₂F₄I, CF₂═CFOCF₂CF(CF₃)OC₂F₄I, CH₂═CHCF₂CF₂I, CF₂═CFOCF₂CF₂CH₂I, CF₂═CFOCF₂CF₂CH₂CH₂I, CF₂═CFOC₄F₈CH₂CH₂I, and combinations thereof.

The fluoropolymer presently disclosed is typically prepared by a sequence of steps, which can include polymerization, coagulation, washing, and drying. In some embodiments, an aqueous emulsion polymerization can be carried out continuously under steady-state conditions. For example, an aqueous emulsion of monomers (e.g., including any of those described above), water, emulsifiers, buffers and catalysts can be fed continuously to a stirred reactor under optimum pressure and temperature conditions while the resulting emulsion or suspension is continuously removed. In some embodiments, batch or semibatch polymerization is conducted by feeding the aforementioned ingredients into a stirred reactor and allowing them to react at a set temperature for a specified length of time or by charging ingredients into the reactor and feeding the monomers into the reactor to maintain a constant pressure until a desired amount of polymer is formed. After polymerization, unreacted monomers are removed from the reactor effluent latex by vaporization at reduced pressure. The fluoropolymer can be recovered from the latex by coagulation.

The polymerization is generally conducted in the presence of a free radical initiator system, such as ammonium persulfate, disodium phosphate heptahydrate, potassium permanganate, AIBN, bis(perfluoroacyl) peroxides, or a combination of two or more thereof. In some embodiments, polymerization is initiated with an initiator system selected from a combination of a fluoroaliphatic sulfinate and an oxidizing agent capable of oxidizing the sulfinate to a sulfonyl radical and/or a combination of a free radical initiator and a chloride salt. Examples of oxidizing agents used as a part of the initiator include, but are not limited to, free radical initiators such as persulfate, a permanganic acid or a salt thereof such as potassium permanganate. Examples of chloride salts used as part of the initiator include, but are not limited to, chloride salts with organic and inorganic cations such as tetrabutyl ammonium chloride and ammonium chloride. One skilled in the art would know initiators that work in similar polymerizations and how to initiate the polymerization to form the fluoropolymer.

The amount of initiator used to initiate the polymerization to for the fluoropolymer can vary. In one embodiment, the amount of initiator used to initiate the polymerization is an initiating effective amount, alternatively from 0.01% to 15% (w/w), alternatively from 0.1% to 10% (w/w), alternatively 0.1% to 5% (w/w), based on the weight of all initiator and monomer, of initiator. An “initiating effect amount” of initiator means an amount sufficient to initiate the polymerization reaction. One skilled in the art would know how to select and use and determine the proper amount of initiator to use to initiate the polymerization.

One skilled in the art would know how, based on the disclosure herein, to optimize the temperature and pressure conditions, select an appropriate reaction vessel, determine monomer, initiator, and other component percentages to perform the polymerization to form the fluoropolymer of the invention.

The fluoropolymer may also be described according the repeat units in the fluoropolymer. In one embodiment, the fluoropolymer comprises a repeating unit according to the formula —(C(R¹)(H)CF₂)— (3), where R¹ is —OCRF₂, where R is H, F, Cl, Br, CF₂H, CF₃, CF₂CF₂H, linear perfluoroalkyl having 1 to 12 carbon atoms, or cyclic perfluoroalkyl having 1 to 12 carbon atoms; alternatively comprises a repeating unit according to formula (3) and further comprises one or more repeating units (4) —(CF₂CF₂)—; (5) —(CH₂—CF₂)—; (6) —(CF₂C(R¹)F)—, where R¹ is —(OCF₂C(F)(CF₃)OCF₂CF₂C═N); and/or (7) —(C(R¹)(F)CF₂)—.

The fluoropolymer comprising the repeating unit (3) and alternatively repeating unit (3) and one or more of repeat units (4), (5), (6), and/or (7) may be made using monomers, process, conditions, and reaction vessels as described above for the fluoropolymer made by polymerizing the halo (alkyl ethyl) ether according to formula (1). One skilled in the art would know how to make the fluoropolymer having the repeating units (3)-(7) based on the disclosure above. In one embodiment, the fluoropolymer comprising the repeating units (3)-(7) is made using the halo (alkyl ethyl) ether made using the method of making the halo (alkyl ether) according to formula (1) described above.

The properties of the fluoropolymer comprising the repeating unit (3) and alternatively repeating unit (3) and one of more of repeat units (4), (5), (6), and/or (7) may have the same properties of the fluoropolymer made by polymerizing with the halo (alkyl ethyl) ether according to formula (1) described above. For example, the fluoropolymer may be curable, non-curable, amorphous, or non-amorphous.

The fluoropolymer may form part of a compound.

An Article, comprising a cured compound that, before curing, comprised: a fluoropolymer made by polymerizing the halo (alkyl vinyl) ether, where the halo (alkyl vinyl) ether and the method of making the halo (alkyl vinyl) ether are as described above. Examples of the article include, but are not limited to, a gasket, seal, tubing, sheet, washer, or O-ring.

An Article, comprising a cured compound that, before curing, comprised: a fluoropolymer. The fluoropolymer is as described above and contains the repeating units also described above. Examples of the article include, but are not limited to, a gasket, seal, tubing, sheet, washer, or O-ring.

The method claimed below allows for the halo (alkyl vinyl) ether to be produced at increased yields over known methods. Therefore, the halo (vinyl ether) produced may be economically produced and used to make new fluoropolymers with potentially improved properties compared to known fluoropolymers.

### EXAMPLES

The following examples are presented to better illustrate the method of the present invention, but are not to be considered as limiting the invention, which is delineated in the appended claims. Unless otherwise noted, all parts and percentages reported in the examples are by weight. The following table describes the abbreviations used in the examples:

TABLE 1
List of abbreviations used in the examples.

| Abbreviation | Word |
|---|---|
| g | gram |
| Me | methyl |
| wt | weight |
| % | percent |
| mol | mole |
| hr or h | hour |
| ° C. | degrees Celsius |
| NA | Not Applicable |
| | milliliter |
| Solids Content | (wt. of dried sample/wt. of initial sample) × 100 and |
| | determined as described below |
| cm | Centimeter |
| DMF | Dimethylformamide |
| NMR | Nuclear Magnetic Resonance |
| MPaG | Megapascal gauge |
| cc | Cubic centimeters |

### Example 1: Preparation of difluoromethyl 2,2-difluoroethenyl ether (CF₂═CHOCHF₂) Via dehalogenation of isoflurane (CF₃C(Cl)(H)OCF₂H)

A one gallon Hastalloy-C autoclave with cooling coil and pressure relief device was charged with anhydrous zinc powder (492.0 g, 7.524 mols) and anhydrous DMF (1480 g). The mixture was flushed thoroughly with nitrogen and isoflurane (1195 g, 6.477 mols) was fed into the reactor. The reactor was heated at 140° C. for 10 h. The contents were then cooled to 15° C. and drained. GC-FID analysis indicated the complete transformation of isoflurane to difluoromethyl 2,2-difluoroethenyl ether. The mixture was filtered cold to remove the excess zinc and zinc salts. The resulting filtrate was distilled under reduced pressure to obtain crude difluoromethyl 2,2-difluoroethenyl ether (840 g) with >95% (w/w) purity contaminated with traces of DMF. The crude product was then redistilled under atmospheric pressure and fractions collected at 17-20° C. were combined to obtain difluoromethyl 2,2-difluoroethenyl ether (759.8 g) as a clear colorless liquid in >99% (w/w) purity. The chemical structure was confirmed by NMR.

### Example 2: Preparation of a TFE-HHPMVE-8-CNVE Copolymer

A polymer containing copolymerized monomers of tetrafluoroethylene (TFE), 2-(difluoromethoxy)-1,1-difluoroethene (HHPMVE), and perfluoro-8 (cyano-5-methyl-3,6-dioxa-1-octene) (8-CNVE) was prepared as follows. 2215 grams of a 2% (w/w) solution of the ammonium salt of CF₃CF₂CF₂OCF(CF₃)CH₂OPO(OH) 2 were loaded into a four-liter, mechanically stirred, water jacketed, stainless steel reactor and oxygen removed by repeated pressurization/depressurization cycles with nitrogen. The nitrogen was removed by repeated pressurization/depressurization cycles with TFE. When the nitrogen content of the headspace was less than 1% (w/w), and the reactor pressure was 0.03 MPaG, 100 ml of HHPMVE were fed to the reactor. The reactor was heated to 80° C. and pressurized to 1.72 MPaG with TFE. 3.6 ml of 8-CNVE were then loaded into the reactor. Forty ml of an initiator solution of 2.5% (w/w) ammonium persulfate and 5.0% (w/w) disodium phosphate heptahydrate were then charged to the reactor to commence polymerization. Additional TFE was fed to the reactor to maintain a pressure of 1.72 MPaG. At 70 grams of TFE added, an additional 50 ml of HHPMVE were fed to the reactor. At 100 grams of TFE added, another 3.6 ml charge of 8-CNVE was loaded to the reactor. At 140 grams of TFE added, an additional 50 ml of HHPMVE were fed to the reactor. After a total of 200 grams of TFE had been fed to the reactor, the reactor was vented to reduce pressure and stop polymerization. A 15.8% (w/w) solids emulsion was obtained from the polymerization. The emulsion was coagulated with potassium aluminum sulfate and dried at 70° C. The resulting polymer had a composition of 71.9 mol % TFE, 27.5 mol % HHPMVE, and 0.6 mol % 8-CNVE by NMR testing. The polymer had a glass transition temperature of 4.0° C. and a melting temperature of 85.8° C.

### Example 3: Preparation of a VF2-HHPMVE Dipolymer

A polymer containing copolymerized monomers of vinylidene fluoride (VF2) and 2-(difluoromethoxy)-1,1-difluoroethene (HHPMVE) was prepared as follows. 2200 grams of deionized water were loaded into a four-liter, mechanically stirred, water jacketed, stainless steel reactor and oxygen removed by repeated pressurization/depressurization cycles with nitrogen. The nitrogen was removed by repeated pressurization/depressurization cycles with VF2. When the nitrogen content of the headspace was less than 1% (w/w), and the reactor pressure was 0.03 MPaG, 52 ml of HHPMVE were fed to the reactor. The reactor was heated to 80° C. and pressurized to 0.83 MPaG with VF2. Twenty ml of an initiator solution of 10% (w/w) ammonium persulfate were then charged to the reactor to commence polymerization. Additional VF2 was fed to the reactor to maintain a pressure of 0.83 MPaG. Twenty-four ml portions of HHPMVE were fed to the reactor after 40, 80, 120, 160, and 200 grams of VF2 had been fed. When a total of 240 grams of VF2 had been fed, the reactor was vented to reduce pressure and stop polymerization. An additional eighty-nine ml of initiator solution were added throughout the polymerization to maintain reaction. A 16.8% (w/w) solids emulsion was obtained from the polymerization. The emulsion was coagulated with potassium aluminum sulfate and dried at 70° C. The polymer had a glass transition temperature of −4.7° C. and a melting temperature of 91.1° C.

### Example 4: Preparation of a TFE-HHPMVE Dipolymer

A polymer containing copolymerized monomers of tetrafluoroethylene (TFE) and 2-(difluoromethoxy)-1,1-difluoroethene (HHPMVE) was prepared as follows. 2215 grams of a 2% (w/w) solution of the ammonium salt of CF₃CF₂CF₂OCF(CF₃)CH₂OPO(OH)₂ were loaded into a four-liter, mechanically stirred, water jacketed, stainless steel reactor and oxygen removed by repeated pressurization/depressurization cycles with nitrogen. The nitrogen was removed by repeated pressurization/depressurization cycles with TFE. When the nitrogen content of the headspace was less than 1% (w/w), and the reactor pressure was 0.03 MPaG, 71.5 ml of HHPMVE were fed to the reactor. The reactor was heated to 80° C. and pressurized to 1.72 MPaG with TFE. Forty ml of an initiator solution of 2.5% (w/w) ammonium persulfate and 5.0% (w/w) disodium phosphate heptahydrate were then charged to the reactor to commence polymerization. Additional TFE was fed to the reactor to maintain a pressure of 1.72 MPaG. When a total of 400 grams of TFE had been fed, the reactor was vented to reduce pressure and stop polymerization. A 21.5% (w/w) solids emulsion was obtained from the polymerization. The emulsion was coagulated with potassium aluminum sulfate and dried at 70° C. The polymer had a melting temperature of +304.4° C.

### Example 5: Preparation of a TFE/HHPMVE/PMVE/8-CNVE Tetrapolymer

A polymer containing copolymerized monomer units of tetrafluoroethylene (TFE), perfluoro(methyl vinyl ether) (PMVE), 2-(difluoromethoxy)-1,1-difluoroethene (HHPMVE), and perfluoro-8(cyano-5-methyl-3,6-dioxa-1-octene) (8-CNVE) was prepared as follows. An aqueous stream was fed continuously to a 1 liter mechanically stirred, water jacketed, stainless steel autoclave at a rate of 235 cc/hr. The stream contained 0.09% (w/w) ammonium persulfate, 0.53% (w/w) disodium phosphate heptahydrate, 3.1% (w/w) of the ammonium salt of CF₃CF₂CF₂OCF(CF₃)CH₂OPO(OH) 2 and 0.5% (w/w) of the ammonium salt of carboxylic acid-terminated poly(hexafluropropylene oxide) (Krytox® 157 FSL). Using a diaphragm compressor, a mixture of TFE (67.4 g/hr) and PMVE (97.5 g/hr) was fed at constant rate. HHPMVE was fed at a rate of 4.5 g/hr and 8-CNVE was fed at a rate of 5.5 g/hr. The temperature was maintained at 80° C. and the pressure at 4.1 MPaG throughout the reaction. The polymer emulsion was removed continuously by means of a letdown valve and the unreacted monomers were vented. The polymer was coagulated with magnesium sulfate heptahydrate and dried at 70° C. The resulting polymer had a composition of 75.1 mol % TFE, 22.5 mol % PMVE, 1.7 mol % HHPMVE, and 0.7 mol % 8-CNVE. The polymer had an inherent viscosity of 1.33 measured in a solution of 0.1 g polymer in 100 g of Flutec® PP-11 (F₂ Chemicals Ltd, Preston, UK) and a Mooney viscosity (1+10) of 92.5 measured at 175° C. The polymer had a glass transition temperature of −0.5° C. and no melting transition.

1. A method for making a halo (alkyl vinyl) ether, comprising the following steps: heating a reaction mixture comprising a combination of
i) a metal;
ii) a solvent; and
iii) a halo (alkyl ethyl) ether according to formula (1) 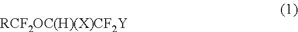
where R is independently H, F, Cl, Br, CF₂H, CF₃, CF₂CF₂H, linear perfluoroalkyl having 1 to 12 carbon atoms, or cyclic perfluoroalkyl having 1 to 12 carbon atoms, and X and Y are independently Cl, Br, I, or F, where X and Y are not both F;
to form a reaction product mixture comprising a halo (alkyl vinyl) ether, the solvent, unreacted metal, and a metal salt.

2. The method according to claim 1, wherein the halo (alkyl vinyl) ether is according to formula (2) 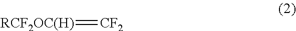
where R is as defined for formula (1).

3. The method according to claim 1, wherein the metal is zinc, magnesium, cadmium, or indium, wherein the solvent is an anhydrous polar aprotic solvent, and wherein the halo (alkyl ethyl) ether is anhydrous isoflurane, and wherein the halo (alkyl vinyl) ether is difluoromethyl 2,2-difluoroethenylether.

4. The method claim 1, further comprising the steps of:
(I) combining the metal and the solvent in a reactor;
(II) flushing the metal and solvent combination with nitrogen; and
(III) adding the halo (alkyl ethyl) ether to the metal and solvent combination to form the reaction mixture; and
(IV) holding the reaction mixture at a temperature from RT ° C. to 160° C. for from 2 to 15 hours.

5. The method according to claim 4, further comprising the step of:
(V) cooling the reaction product mixture to a temperature from 2° C. to 20° C.; and
(VI) filtering the reaction product mixture to remove the metal and metal salts to form a filtered reaction product mixture comprising the solvent and the halo (alkyl vinyl) ether; and
(VII) purifying the halo (alkyl vinyl) ether.

6. The method according to claim 5, wherein the purifying is by distillation.

7. The method according to claim 1, wherein the mole ratio of metal to halo (alkyl ethyl) ether in the reaction mixture is from 1:0.5 to 1:1.5.

8. A fluoropolymer made by polymerizing the halo (alkyl vinyl) ether made by the process of claim 1, wherein the fluoropolymer is curable.

9. The fluoropolymer according to claim 8, wherein the halo (alkyl vinyl) ether is polymerized with one or more additional monomers.

10. The fluoropolymer according to claim 9, wherein the additional monomers are one or more monomers selected from the group consisting of tetrafluoroethylene (TFE), vinyl fluoride (VF), a perfluoro(alkyl vinyl) ethers (PAVE), ethylene, tetrafluoropropene (TFP), ester vinyl ether, methyl perfluoro(5-methyl-4,7-dioxanon-8-enoate) (EVE), perfluoro(4-methyl-3,6-dioxaoct-7-ene)sulfonyl fluoride (PSEPVE), vinylidene fluoride (VF2), hexafluoropropylene (HFP), chlorotrifluoroethylene (CTFE), propylene (P), MOVE, pentafluoropropene (HPFP), perfluoro(3-methoxypropyl vinyl) ether (MV-31), and their analogs.

11. The fluoropolymer according the claim 10, wherein the perfluoro(alkyl vinyl) ether is perfluoromethylvinylether (PVME), perfluoro(ethyl vinyl) ether (PEVE), perfluoro(propyl vinyl) ether (PPVE) or a long chain perfluorovinyl ether.

12. The fluoropolymer according to claim 8, wherein the halo (alkyl vinyl) ether is further polymerized with a cure site monomer.

13. The fluoropolymer according to claim 12, wherein the cure site monomer is perfluoro(8-cyano-5-methyl-3,6-dioxaoct-1-ene), CF₂═CF—O(CF₂)ₙCN (linear CNVE), CF₂═CF—O[CF₂—CFCF₃—O]ₙ—CF₂—CFCF₃CN, or CF₂═CF—[OCF₂CFCF₃]ₓ—O—(CF₂)ₙCN, CF₂═CF—O—(CF₂)ₙ—O—CF(CF₃)CN.

14. A fluoropolymer, comprising:
a. a repeating unit (I),
(I) (C(R¹)(H)CF₂)—, where R¹ is —OCRF₂, where R is H, F, Cl, Br, CF₂H, CF₃, CF₂CF₂H, linear perfluoroalkyl having 1 to 12 carbon atoms, or cyclic perfluoroalkyl having 1 to 12 carbon atoms.

15. The fluoropolymer according to claim 14, further comprising
b. a repeating unit of one or more of formulas (II), (III), (IV), or (V):
—(CF₂CF₂)—; (II)
—(CH₂—CF₂)—; (III)
—(CF₂C(R¹)F)—, where R¹ is —(OCF₂C(F)(CF₃)OCF₂CF₂C≡N); (IV)
—(C(R¹)(F)CF₂)—. (V)

16. The fluoropolymer according to claim 8, wherein the fluoropolymer is curable.

17. An Article, comprising a cured compound that, before curing, comprised: a fluoropolymer made by polymerizing the halo (alkyl vinyl) ether made by the process of claim 1.

18. The article of claim 17 in the form of a gasket, seal, tubing, sheet, washer, or O-ring.

19. An Article, comprising a cured compound that, before curing, comprised: a fluoropolymer according to claim 14.
